(19) United States
(12) Patent Application Publication
(10) Pub. No.: US 2004/0219191 A1
(43) Pub. Date: Nov. 4, 2004

(54) Use of a dopamine agonist with a short half-life for treating illnesses which can be treated by dopaminergic means

(76) Inventor: Karsten Kuhn, Berlin (DE)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/450,483

(22) PCT Filed: Aug. 24, 2001
(86) PCT No.: PCT/EP01/09822

(30) Foreign Application Priority Data
Dec. 16, 2000 (DE) 100 64 453.8

(51) Int. Cl. A61K 31/198; A61K 9/70

(52) U.S. Cl. 424/449; 514/567

(57) ABSTRACT

The invention relates to the use of a dopamine agnostic active ingredient with a short half-life in the form of a transdermal therapeutic system (TTS) for treating illnesses which can be treated by dopaminergic means.

The present invention relates to the use of a means including a transdermal therapeutic system (TTS) with a dopamine agonistic agent with a short half-life for treating dopaminergic diseases in that the TTS is applied daily at bedtime.

The term “TTS” mostly denotes percutaneously acting but also transmucosally acting systems. A TTS typically has a sheet-like structure and is attached to an area of the skin. A TTS mostly includes a matrix containing an active ingredient (optionally in the form of a salt) and/or an active ingredient reservoir, and a diffusion barrier that is permeable to the active ingredient on the skin side of the active ingredient reservoir. The system can optionally be attached to the skin by an additional skin-side adhesive that is permeable to the active ingredient. Alternatively, the matrix and/or diffusion barrier can itself have adhesive properties. And finally a non-adhesive TTS can be attached to the skin using other auxiliary means such as adhesive tapes or bandages. The matrix is a material in which the active ingredient is immobilized. An active agent in an active ingredient reservoir however is not necessarily immobilized, which is why the active ingredient reservoir must be enclosed. The diffusion barrier forms the skin-side portion of this shell. It goes without saying that all other parts of the shell should be as impermeable as possible, including diffusion paths, to the active ingredient. Immobilized means in this context that any uncontrolled active ingredient flow is prevented. However diffusion of an active agent in a matrix and/or through a diffusion barrier is not only possible but intended. The diffusion coefficients eventually determine the active ingredient flux from the TTS into a patient's skin. The dose released into a patient's skin is in first approximation a linear function of the active area of the TTS. The active area is the contact area of those TTS portions that allow active ingredient diffusion.

A TTS designed as described above with lisuride as the active ingredient and its use for treating Parkinson's disease are known in principle from publication WO 92/20339. It specifically describes the effect of propylene glycol lauric acid on the flux, i.e. a considerable increase in flux. A TTS containing lisuride is further known from publication WO 91/00746.

Parkinson's disease and other disorders for which a dopaminergic therapy is indicated are severe chronic and disabling diseases that are frequently treated in today's clinical practice by administering a combination of dopaminergic substances orally. These typically include various formulations of levodopa (high initial flux rate, normal or slow release), levodopa boosters (such as decarboxylase inhibitors as the base and optionally COMT inhibitors or MAO-B inhibitors), and various dopamine agonists such as bromocriptine, lisuride, cabergoline, ropinirole, pramipexole, pergolide as well as amantadines and, occasionally, anticholinergic agents. The pharmacokinetics of fast-acting levodopa is hard to control for various reasons, and dopamine agonists do not allow safe bioavailability and thus efficacy predictions.

Either a continuous or a discontinuous stimulation may be required depending on the stage of the disease and the actual status of the patient. A good foundation is laid when the level of dopaminergic agents is kept stable across the entire day. However patients frequently report that they often need to take a fast-acting dopaminergic agent especially in the morning or at certain times of the day to overcome acute motoric disturbances, severe and painful dystonia, etc. (“kick”). In extreme cases, such sudden “off” states of motoric performance and akinesia (sometimes predictable early in the morning or afternoon, but frequently all of the sudden and unexpectedly) can only be controlled with injectable active agents such as apomorphine. On the other hand, strong and fast efficacy hikes can cause disturbing side effects (e.g. nausea, emesis, orthostatic hypotension, narcoleptic attacks). Overdoses due to the narrow therapeutic time window of all these dopaminergic agents can result in severe dyskinesia, dystonia or, especially after long-term treatment with levodopa and/or agonists (in older patients), psychoses. The latter severe problem is mainly connected with high active agent concentrations in the plasma over night that are known to destroy regular sleeping patterns and to prevent the REM sleep phase (with REM rebound during daytime as an indication of a psychosis). Patients must therefore often be adapted to side effects over several weeks as indoor patients in more or less specialized hospitals.

It is the technological problem of the invention to provide an agent and a treatment plan for treating dopaminergic diseases while preventing or at least reducing disturbing side effects, controlling the initial flux rate of the active agent and keeping good control of the agent due to its short half-life.

The invention solves this technological problem by using a dopamine agonist with a short half-life in the form of an agent comprising at least one composition in two discrete doses, of which one is a transdermal therapeutic system (TTS) containing the dopamine agonistic agent with a short half-life for the treatment of dopaminergically treatable diseases, said TTS being replaced daily at bedtime. More specifically, the invention specifies the use of a dopamine agonistic agent with a short half-life in the form of at least two discrete compositions consisting a) of a transdermal therapeutic system (TTS) that contains the dopamine agonistic agent with a short half-life and b) of one or more other preparation(s) containing L-DOPA and, optionally, a decarboxylase inhibitor for treating dopaminergically treatable diseases and suitable for oral administration, said TTS being replaced at bedtime.

The dopaminergic therapy is specifically suited for treating diseases from the “parkinsonism, particularly Parkinson's disease” group.

Doses as high as possible are desirable for treating Parkinson's disease; up to now, these doses have to be given orally throughout the day or in the form of an agonist with a very long half-life. Oral administration throughout the day entails the problem of pulsatile stimulation while an agonist with a long half-life has a potential of agent accumulation and the resulting loss in efficacy (receptor desensitization) and/or, after long-term treatment, overstimulation in the clinical picture of psychosis. A transdermal therapeutic system also improves compliance, which is of critical importance for any combinatory treatment of this disease and the mostly older patients. Better control as well as nighttime application of the patch (night patch at bed time, approx. 10.00 PM) open a wide therapeutic window for treating parkinsonism.

The unprecedented advantages of applying/replacing a patch containing a dopamine agonistic active ingredient with a short half-life at bedtime are the following:

Continuous stimulation with a low risk of desensitization and the resulting need to increase doses to reach the desired therapeutic effect

Reduces the patients' akinesia at night (turning over while asleep) and thus increases the quality of sleep

Reduces akinesia in the morning and thus increases the patients' quality of life (self-reliance)

Prevents psychoses caused by permanent stimulation and provides a way for emergency measures (patch removal) in the event of a psychosis

Reduces dyskinesia in a combinatory treatment, e.g. with L-Dopa.

The dopamine agonistic active ingredient can be in the form of a free base or a physiologically tolerable salt. Suitable salts include sulfates, phosphates, maleates, citrates and succinates, particularly hydrogen maleate.

The dopamine agonistic active agent is preferably an ergoline derivative of the formula I or its physiologically tolerable thereof salt with an acid

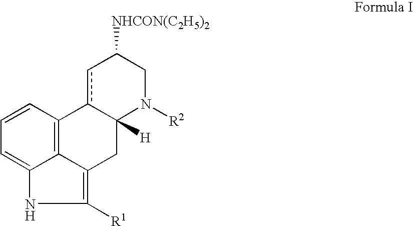

where [symbol] is a single or double bond wherein R¹ is an H atom or a halogen atom, particularly a bromine atom, and wherein R² is C1-4 alkyl, particularly methyl.

The list of ergoline derivatives that can be used particularly includes the following: Lisuride, bromolisuride (3-(2-bromo-9,10-didehydro-6-methyl-8α-ergolinyl)-1,1-diethyl urea), terguride (3-(6-methyl-8α-ergolinyl)-1,1-diethyl urea) and proterguride (3-(6-propyl-8α-ergolinyl)-1,1-diethyl urea). However it is preferred when the ergoline derivative is lisuride (3-(9,10-didehydro-6-methyl-8α-ergolinyl)-1,1-diethyl urea) or its physiologically compatible salt with an acid. The production of lisuride and other suitable ergolines according to the invention is described, inter alia, in U.S. Pat. No. 3,953,454, EP 056 358 and U.S. Pat. No. 4,379,790.

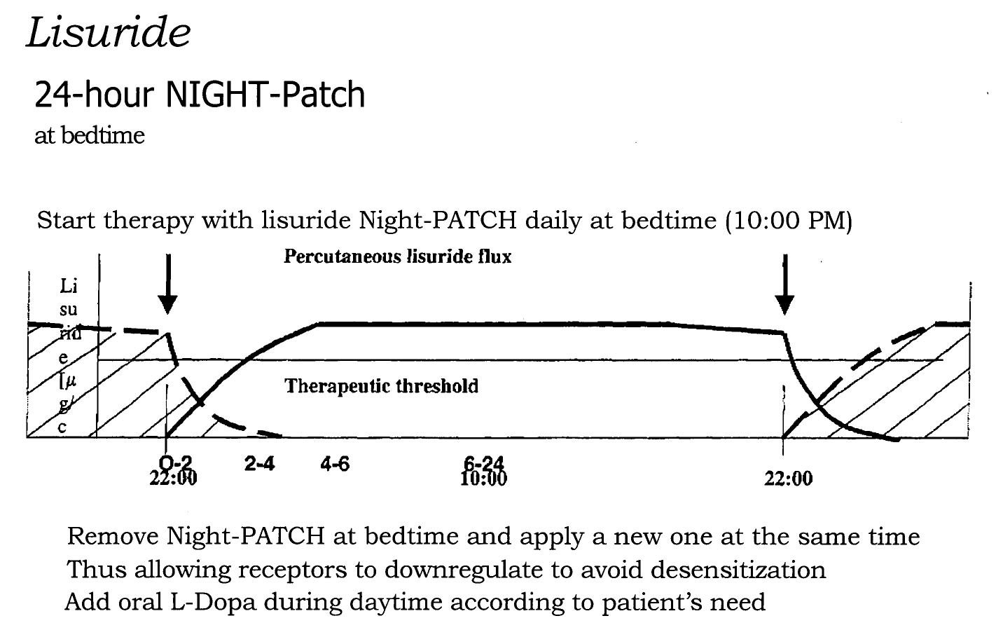

An innovative and surprising finding of applying/replacing the lisuride-containing night patch at bedtime is that the relatively slow rise in flux until a constant value is reached (the total active agent dose being defined by the size of the patch) and the short half-life of lisuride in the plasma results in a drop below the therapeutic threshold for 1 to 3 hours (lag time) at night after the patch has been changed (t½=1 to 2 hours). The clinical benefit of such a time lag is that for the first-time it addresses the potential desensitization (loss in efficacy) caused by permanent receptor stimulation. These receptors normalize during this time and thus become sensitive enough for another stimulation from the next lisuride night patch. This temporary drop below the therapeutic threshold is clinically justifiable at night (after midnight in the present case). At daytime, such drop would result in unjustifiable restrictions of the patients' mobility for 2 to 3 hours (FIG. 1). Dopamine agonists with a longer half-life (up to 21 hours terminal half-life) result in agent accumulation and loss in efficacy at the receptors. Another advantage of the use according to the invention is a constant flux through the skin due to the good water solubility of lisuride or a salt thereof.

The ergoline derivatives of the formula I have a partially dopamine agonistic or partially antagonistic effect that contributes to preventing the development of psychoses and can improve existing psychoses and similar problems.

If required, e.g. at an advanced state of the disease, treatment is supplemented by administering oral preparations of L-DOPA, optionally in combination with decarboxylase inhibitors such as benserazide or carbidopa. Motoric blockages and akinesia are removed whenever required by such oral administration and the fast extra action as needed.

More specifically, it is preferred that the TTS comprises a pharmaceutical layer containing at least one matrix that contains an active ingredient and/or an active ingredient reservoir and a diffusion barrier through which the active ingredient can permeate on the skin side of the TTS and, as the active ingredient with a short half-life, an ergoline derivative of the formula I or a physiologically tolerable salt thereof with an acid, said ergoline derivative having a half-life in the range from 0.5 to 4 hours, particularly from 1 to 2 hours, so that a drop below the therapeutic threshold for 1 to 3 hours can be achieved. The matrix and/or diffusion barrier can be selected so that the transdermal flux F through human skin measured as described in Example 1 is in the range from 0.1 to 5.0 μg/cm²/h. It is preferred to select F and the active surface area to achieve levels in the plasma from 0.1 to 2 ng/ml.

It is preferred that the ergoline derivative is a lisuride base. A covering layer can be arranged on the side of the matrix and/or active ingredient reservoir facing away from the skin.

The preparation in tablet form prepared for oral administration preferably contains 25 to 1,000 μg of L-DOPA (per tablet).

The quantities of each active ingredient per day in relation to the neutral compound in an oral administration are 50 to 700 mg/day for L-DOPA, 12.5 to 200 mg/day for benserazide and 25 to 175 mg/day for carbidopa.

The TTS can be designed as follows. A covering layer can be arranged on the side of the matrix and/or active ingredient reservoir facing away from the skin. It may be formed by films of polyethylene or polyester. It is typically 10 to 100 microns in thickness. The covering layer may be pigmented, varnished, and/or metal plated to ensure sufficient protection from light. Metal plating involves applying a very thin layer (typically less than 1 micron, mostly in the 10-100 nm range) of a metal such as aluminum to the covering layer. Pigments can be all pigments commonly used for coating including effect pigments as long as these are physiologically harmless. A detachable liner such as a siliconized or fluoropolymer-coated protective film can be provided on the application side.

The matrix and/or diffusion barrier may comprise as their main matrix component a substance selected from the group consisting of “polyacrylate, polyurethane, cellulose ether, silicone, polyvinyl compounds, polyisobutylene compounds, silicate and mixtures of these substances as well as copolymers of these polymeric compounds,” preferably polyacrylate. A main matrix component makes up at least 50 percent by weight, e.g. at least 80-90 percent by weight of the matrix (matrix to be understood as the finished layer, i.e. main matrix component(s) with adjuvant(s) and active ingredient(s)). The desired flux is set by selecting the substance depending on the diffusion coefficient of the active ingredient and, if required, by selecting the layer thickness of the matrix in orthogonal direction to the skin surface. Matrix thickness is typically in the range from 10 microns to 500 microns.

A preferred polyacrylate adhesive as the main matrix component is commercially available under the brand name GELVA® multipolymer solution 7881, provided by Monsanto Deutschland GmbH, Düsseldorf. We expressly refer to the product sold under this name and its datasheet in the version of Apr. 23, 1996. Another suitable product is Eudragit® E100 provided by Röhm, Germany.

The polyacrylate adhesives listed above provide an advantageous non-trivial combination of properties, namely optimum flux, good adhesive power, good skin compatibility, and durability.

The diffusion barrier can alternatively comprise as its main barrier component a polymer selected from the group consisting of “cellulose ester, cellulose ether, silicone, polyolefin and mixtures as well as copolymers of these substances.” What has been said above about the term of the main matrix component analogously applies to the term of the main barrier component. The diffusion barrier can be a film with a thickness from 10 microns to 300 microns; the actual film thickness is selected (in conjunction with the diffusion coefficient of the active ingredient in the polymer) according to the desired flux.

The matrix and/or active ingredient reservoir and/or diffusion barrier may contain the common adjuvants used in TTSs. It is preferred to use a penetration-enhancing agent that is preferably selected from the group consisting of “C1-C8 aliphatic, cycloaliphatic and aromatic alcohols, saturated and unsaturated C8-18 fatty alcohols, saturated and unsaturated C8-18 fatty acids, hydrocarbons and hydrocarbon mixtures, fatty acid esters from C3-19 fatty acids and C1-6 alkyl monools, dicarboxylic acid diesters from C4-8 dicarboxylic acids and C1-6 alkyl monools, and mixtures of these substances. Penetration-enhancing agents improve the flux of the active ingredient through the skin to which the TTS is attached. Examples of the substances listed above are: 1,2-propane diol, menthol, dexpanthenol, benzyl alcohol, lauryl alcohol, isocetyl alcohol, cetyl alcohol, mineral oil, lauric acid, isopalmitic acid, isostearic acid, oleic acid; methyl ester, ethyl ester, 2-hydroxyethyl ester, glycerol ester, propyl ester, isopropyl ester, butyl ester, sec. butyl ester or isobutyl ester of lauric acid, myristic acid, stearic acid, or palmitic acid. Use of dimethyl isosorbide, isopropyl myristate and lauryl alcohol is preferred, use of lauryl alcohol is most preferred. Other suitable adjuvants include crystallization inhibitors. Suitable crystallization inhibitors are highly dispersed silicon dioxide or macromolecular substances such as polyvinyl pyrrolidone, polyvinyl alcohols, dextrines, sterines, bile acids and, in particular, polyvinyl pyrrolidone vinylacetate copolymers such as Kollidon® VA 64.

It goes without saying that the penetration-enhancing agent has to be able to sufficiently diffuse through the matrix or diffusion barrier. If a matrix and lauryl alcohol as an adjuvant are used, it is preferred that the lauryl alcohol makes up 10 to 30 percent by weight, most preferred 15 to 20 percent by weight, of the matrix.

The adjuvants can basically make up from 0 to 50 percent by weight of the matrix. The active ingredient can make up 0.5 to 20 percent by weight, preferably 1 to 10 percent by weight, of the matrix. The sum total of main matrix component, adjuvants and active ingredients is always 100 percent by weight.

The active ingredient dose in a human body carrying a TTS is dependent on the diffusion-related properties of the TTS mentioned above and also on its active surface area on the skin. Active surface area means the area over which the matrix or diffusion barrier comes to rest on the skin. Variation in accordance with the desired dosage will preferably be in a range from 1 to 100 cm². Within the scope of this invention, a physician can easily set up personalized dose variations for a flux adjusted to the given indication by selecting a suitable patch size. Thus the treatment can easily be adjusted to different body weights, age groups, etc. It is particularly feasible to equip a TTS comprising a (rather large) standard area with subdivision markers for partial doses so that a user can just separate and use a partial area corresponding to the specified dose. The respective subsections can easily be printed on the covering layer.

The invention will be explained in more detail below based on various non-limiting examples.

### EXAMPLE 1

### Flux Measurement

A FRANZ flow-through diffusion cell is used for flux measurement. The measured area is 2 cm². 4 cm² of ventral and dorsal skin of a male hairless mouse (MF1 hr/hr Ola/Hsd, provided by Harlan Olac, UK) are used as skin sample after carefully removing any subcutaneous fatty tissue. A 2 cm² TTS is applied to the skin sample. The acceptor medium is placed on the opposite side. It is diluted HHBSS (Hepes Hanks Balanced Salt Solution) containing 5.96 g/l of Hepes, 0.35 g/l of NaHCO₃ and 0.1 ml/l 10× of HBSS (provided by Gibco, Eggenstein, Del.). Furthermore, 1000 I.U./ml of penicillin (benzylpenicillin potassium salt, provided by Fluka, Neu-Ulm, Del.) are used.

The flux is measured as described below. First, the TTS to be measured is applied to the skin. The skin is installed in the diffusion cell immediately thereafter. Samples of the acceptor medium are taken at 2-hour intervals between t=0 hrs and t=6 hrs and at 8-hour intervals between t=6 hrs and t=54 hrs. 1 ml of acceptor medium per hour is pumped through the diffusion cell using a peristaltic pump. The temperature of the acceptor medium is controlled using a circulating water bath which keeps the skin at a temperature of 31° C. with an accuracy of 1° C.

The active ingredient concentration in the acceptor medium is determined in accordance with the following specifications using a radioimmunoassay.

Calibration curves: These are constructed using two different methanol solutions of non-radioactive lisuride hydrogen maleate salt, each containing 1 mg/ml. These solutions are individually diluted with BSA buffer (0.041 M of Na₂HPO₂*2H₂O, 0.026 M of KH₂PO₄, 0.154 M of NaCl, 0.015 M of NaN₃, 0.1% (w/v) of BSA, pH 7, supplemented with 0.05% (w/v) of ascorbic acid) to obtain lisuride free base concentrations in the range from 1,000-3.9 pg/0.1 ml. In addition, a sample without active ingredient (0 pg) is used. The calibration samples are analyzed three times. The lisuride concentrations are calculated using the pharmacokinetic PC program RIO 2.5 (other common software may also be used).

Sample preparation: The acceptor medium is diluted with BSA buffer prior to the analysis to set the concentrations to an analyzable range of the calibration curve. 100 μl of diluted sample are directly subjected to radioimmunological analysis.

Antiserum: The antiserum (rabbit) is obtained by immunizing with lisuride-1-succinyl-BSA, an immunogen. The antiserum in the assay is diluted 1:12,500.

Tracer: ³H-lisuride hydrogen maleate with a specific activity of 4.3 GBq/mg is used.

Incubation: 0.1 ml of BSA buffer with active ingredient, 0.1 ml of tracer solution (ca. 5000 cpm/0.1 ml of BSA buffer) and 0.1 ml of diluted antiserum (1:12,500) are added to 0.7 ml of BSA buffer and incubated for 18 hours at 4° C.

Separation: antibody-bound lisuride is separated from free lisuride by adding 0.2 ml of charcoal suspension (1.25%(w/v) and 0.125% (w/v) dextrane in BSA buffer) and incubation for 30 minutes at 0° C. The charcoal is sedimented by centrifuging for 15 minutes at 3,000 g. The supernatant liquid (containing antibody-bound active ingredient) is decanted and subjected to radiometric analysis.

Radiometric analysis: 4 ml of Atomlight (NEN) scintillation cocktail are added to the supernatant. The count is carried out using a WALLAC 1409 or 1410 β-scintillation counter without quench control.

Analysis: The percutaneous skin flux is calculated as follows:

F=(C*R)/(A*T),

where F is the percutaneous flux [ng/cm²/h], C the active ingredient concentration in the acceptor medium [ng/ml], R the acceptor medium flow [1 ml/h], A the measured area [2 cm²] and T the sample-taking interval [h].

The maximum transdermal active ingredient flux is obtained directly from the data. Mean percutaneous flux values are determined during days 1 and 2 of the experiment based on the cumulative absorbed dose in time intervals t=0-22 and t=22-54.

### EXAMPLE 2

### Manufacturing of a TTS A

15 mg of Kollidon VA 64 (crystallization inhibitor) are dissolved in 15 mg of isopropanol. Then 5 mg of lisuride are sprinkled in. 80 mg of polyacrylate adhesive (Gelva 7881) are placed in a beaker, and the above suspension is added while rerinsing with 30 mg of isopropanol. The crystal-free wet mix obtained is thoroughly intermixed and spread on a siliconized liner using a 500 micron blade. The product is dried at 60° C for 20 minutes, and finally a covering layer is laminated onto it.

Flux measurements as described in Example 1 showed an F value of 0.43 on day 1, 0.44 on day 2, and a maximum F value of 0.85 (each in μg/cm²/h).

### EXAMPLE 3

### Manufacturing of a TTS B

12.5 mg of dimethyl isosorbide are suspended with 2 mg of lisuride in 15 mg of isopropanol. 80 mg of polyacrylate adhesive (Gelva 7881) are placed in a beaker, and the above suspension is added while rerinsing with 30 mg of isopropanol. The crystal-free wet mix obtained is thoroughly intermixed and spread on a siliconized liner using a 500 micron blade. The product is dried at 60° C. for 20 minutes, and finally a covering layer is laminated onto it.

Flux measurements as described in Example 1 showed an F value of 0.23 on day 1, 0.28 on day 2, and a maximum F value of 0.50 (each in μg/cm²/h).

### EXAMPLE 4

### Manufacturing of a TTS C

27.2 mg of Kollidon VA 64 (crystallization inhibitor) and 16.3 mg of lauryl alcohol are dissolved at 60° C. Then 2 mg of lisuride are dissolved in this solution at 60° C. 39.38 mg of Eudragit E100, 13.41 mg of Citroflex 4A and 1.71 mg of succinic acid are molten at 150-200° C. The lisuride solution is added after the batch has cooled down to 80° C. The product is spread at 80° C on a siliconized liner using a 500 micron blade. Then the product is cooled down to 20° C.; optionally, a covering layer may be laminated onto it.

Flux measurements as described in Example 1 showed an F value of 0.90 on day 1, 1.76 on day 2, and a maximum F value of 2.53 (each in μg/cm²/h).

1. Use of a dopamine agonist with a short half-life in the form of an agent comprising at least one composition in two spatially discrete doses, of which one is a transdermal therapeutic system (TTS) containing the dopamine agonistic agent with a short half-life for the treatment of dopaminergically treatable diseases, said TTS being replaced daily at bedtime.

2. Use of a dopamine agonistic agent with a short half-life in the form of at least two discrete compositions consisting a) of a transdermal therapeutic system (TTS) that contains the dopamine agonistic agent with a short half-life and b) of one or more other preparation(s) containing L-DOPA and, optionally, a decarboxylase inhibitor for treating dopaminergically treatable diseases and suitable for oral administration, said TTS being replaced daily at bedtime.

3. The use according to claims 1 or 2 wherein the dopaminergically treatable disease is a disease from the “Parkinson's disease or parkinsonism” group.

4. The use according to any one of claims 1 through 3 wherein the dopamine agonist of the transdermal therapeutic system is an ergoline derivative according to formula 1 or a physiologically compatible salt thereof, 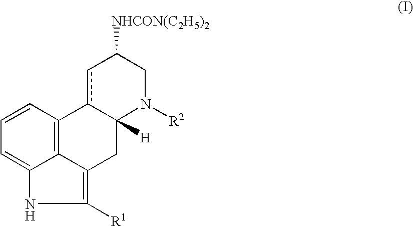
where [symbol] is a single or double bond wherein R1 is an H atom or a halogen atom, particularly a bromine atom, and wherein R2 is C1-4 alkyl.

5. The use according to any one of claims 1 through 4 wherein the active dopamine agonist is lisuride base or a physiologically tolerable salt thereof.

6. The use according to any one of claims 1 through 5 wherein the active dopamine agonist has a half-life of 0.5 to 4 hours, preferably 1 to 2 hours.

7. The use according to any one of claims 1 through 6 wherein the TTS comprises a pharmaceutical layer comprising at least one matrix containing an active ingredient and/or an active ingredient reservoir, and a diffusion barrier on the skin side of said active ingredient reservoir that is permeable to said active ingredient.

8. The use according to claim 7 wherein the matrix and/or diffusion barrier are selected so that the transdermal flux F through human skin is in the range from 0.1 to 5.0 μg/cm²/h.

9. The use according to claim 7 wherein a drop below the therapeutic threshold occurs for 1 to 4 hours when the patch is replaced.

10. The use according to any one of claims 7 through 9 wherein the matrix and/or diffusion barrier comprise as their main matrix component a substance selected from the group consisting of “polyacrylate, polyurethane, cellulose ether, silicone, polyvinyl compounds, polyisobutylene compounds, silicate and mixtures of these substances as well as copolymers of these polymeric compounds.”

11. The use according to any one of claims 7 through 10 wherein the diffusion barrier comprises as its main barrier component a synthetic polymer selected from the group consisting of “cellulose ester, cellulose ether, silicone, polyolefin and mixtures as well as copolymers of these substances.”

12. The use according to any one of claims 7 through 11 wherein the matrix and/or the active ingredient reservoir and/or the diffusion barrier contain a penetration-enhancing agent that is preferably selected from the group consisting of “C1-C8 aliphatic, cycloaliphatic and aromatic alcohols, saturated and unsaturated C8-18 fatty alcohols, saturated and unsaturated C8-18 fatty acids, hydrocarbons and hydrocarbon mixtures, fatty acid esters from C3-19 fatty acids and C1-6 alkyl monools, dicarboxylic acid diesters from C4-8 dicarboxylic acids and C1-6 alkyl monools, and mixtures of these substances.”

13. The use according to claim 2 wherein the preparation meant for oral administration is applied in combination with another preparation for oral administration that contains a decarboxylase inhibitor.

14. The use according to claim 13 wherein the decarboxylase inhibitor is benserazide or carbidopa.
